(19) United States
(12) Patent Application Publication
(10) Pub. No.: US 2024/0408208 A1
(43) Pub. Date: Dec. 12, 2024

(54) Homeopathic mixture for relieving general fatigue symptoms and cancer treatment related fatigue symptoms

(71) Applicant: Yakove Freed

(72) Inventor: Yakove Freed

(21) Appl. No.: 18/207,184

(22) Filed: Jun. 8, 2023

(51) Int. Cl. A61K 41/00; A61K 33/24; A61K 33/42; A61K 33/44; A61K 51/00; A61N 5/10

(52) U.S. Cl. CPC A61K 41/0004; A61K 33/24; A61K 33/42; A61K 33/44; A61K 51/00; A61N 5/1048

(57) ABSTRACT

A homeopathic mixture for relieving fatigue symptoms or cancer treatment related fatigue symptoms or radiation fatigue symptoms that includes cadmium sulphuratum, x-ray, phosphoricum acidum, radium bromatum, and carbo animalis. The cadmium sulphuratum is diluted not less than twelve times and not more than two hundred times, the x-ray is diluted not less than three times and not more than thirty times, the phosphoricum acidum is diluted not less than twelve times and not more than two hundred times, the radium bromatum is diluted not less than twelve times and not more than two hundred times, and the carbo animalis is diluted not less than twelve times and not more than two hundred times, all on a scale of 1:100.

### TECHNICAL FIELD

The present invention refers to method and homeopathic mixture for relieving fatigue symptoms, cancer treatment related fatigue symptoms and radiation fatigue symptoms.

### BACKGROUND ART

Fatigue symptoms are now considered a serious problem that many people suffer from, due to lifestyle and medical problems. In addition to that, cancer treatment related fatigue symptoms considered too as a serious problem. The present invention comes to provide a good solution for alleviating and relieving such fatigue.

### DESCRIPTION OF THE DRAWINGS

The intention of the drawings attached to the application is not to limit the scope of the invention and its application. The drawings are intended only to illustrate the invention and they constitute only one of its many possible implementations.

FIG. 1 illustrates a subject (100).

FIG. 2 illustrates the homeopathic mixture (200) and its ingredient.

### THE INVENTION

Homeopathy is a form of complementary method in which practitioners treat patients using highly diluted ingredients that causes the body to experience low level manifestations of the symptoms that it is trying to eliminate. It is believed that the homeopathic ingredients modify the body's response by enabling the body's defense and healing mechanisms. Because of their long standing use in the United States, the U.S. Congress passed a law in 1938 declaring that homeopathic remedies are to be regulated by the FDA in the same manner as nonprescription, over-the-counter drugs.

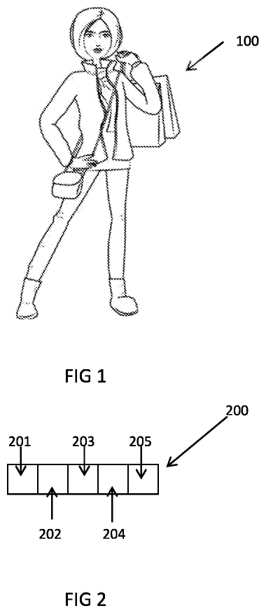

The main object of the present invention is to provide a homeopathic mixture (200) for relieving fatigue symptoms (including radiation fatigue symptoms and cancer treatment related fatigue symptoms) in a subject (100) in need thereof, that comprises: (a) cadmium sulphuratum (201), (b) x-ray (202), (c) phosphoricum acidum (203), (d) radium bromatum (204), and (e) carbo animalis (205). FIG. 1 illustrates a subject (100), and FIG. 2 illustrates the homeopathic mixture (200) and its ingredient.

Cadmium sulphuratum, x-ray, phosphoricum acidum, radium bromatum, and carbo animalis are homeopathic ingredients that are known to be effective in the treatment of variety of medical (physical and emotional) problems. The X-Ray dilution is a homeopathic compound that is prepared by exposing a vial of alcohol to X-rays.

The English letter CH (sometimes in short C) that comes after a number beside the name of the homeopathic ingredient indicates that this ingredient has been diluted on a scale of 1:100. The numeral tells how many times the ingredient was diluted. For example, 30 C means that the active ingredient was diluted to a 1/100 concentration thirty times.

It is desirable that the ingredients of the homeopathic mixture be diluted in the amplitude of the following options to achieve an optimal result:

The cadmium sulphuratum should be diluted not less than twelve times and not more than two hundred times, on a scale of 1:100. Although it is better to dilute it thirty times to achieve an optimal and excellent result.

The said x-ray should be diluted not less than three times and not more than thirty times, on a scale of 1:100. Although it is better to dilute it six times to achieve an optimal and excellent result.

The phosphoricum acidum should be diluted not less than twelve times and not more than two hundred times, on a scale of 1:100. Although it is better to dilute it thirty times to achieve an optimal and excellent result.

The radium bromatum should be diluted not less than twelve times and not more than two hundred times, on a scale of 1:100. Although it is better to dilute it thirty times to achieve an optimal and excellent result.

The carbo animalis should be diluted not less than twelve times and not more than two hundred times, on a scale of 1:100. Although it is better to dilute it thirty times to achieve an optimal and excellent result.

Another object of the present invention is to provide a method for relieving fatigue symptoms ((including radiation fatigue symptoms and cancer treatment related fatigue symptoms) in the subject in need thereof, which comprises administering to the subject and effective amount of the homeopathic mixture that comprises the cadmium sulphuratum, the x-ray, the phosphoricum acidum, the radium bromatum, and the carbo animalis, at the same degrees of dilutions as stated above.

The homeopathic mixture can be solid (tablets) to be dissolved in the mouth of the subject or liquid (drops) and taken orally. The ratio of the quantities of the ingredients of the homeopathic mixture can be varied, although in order to achieve an effective result it is desirable that each ingredient make up a fifth of the mixture. The homeopathic mixture of the present invention was given to many patients during their radiation treatments, and the results showed a great relief in the fatigue symptoms, and a sharp increase in appetite and weight.

What is claimed is:

1. A homeopathic mixture for relieving fatigue symptoms or cancer treatment related fatigue symptoms or radiation fatigue symptoms, that comprises: (a) cadmium sulphuratum, (b) x-ray, (c) phosphoricum acidum, (d) radium bromatum, and (e) carbo animalis.

2. The homeopathic mixture of claim 1,
wherein said cadmium sulphuratum has been diluted not less than twelve times and not more than two hundred times, on a scale of 1:100;
wherein said x-ray has been diluted not less than three times and not more than thirty times, on a scale of 1:100;
wherein said phosphoricum acidum has been diluted not less than twelve times and not more than two hundred times, on a scale of 1:100;
wherein said radium bromatum has been diluted not less than twelve times and not more than two hundred times, on a scale of 1:100; and
wherein said carbo animalis has been diluted not less than twelve times and not more than two hundred times, on a scale of 1:100.

3. The homeopathic mixture of claim 1,
wherein said cadmium sulphuratum has been diluted thirty times on a scale of 1:100;
wherein said x-ray has been diluted six times on a scale of 1:100;
wherein said phosphoricum acidum has been diluted thirty times on a scale of 1:100;
wherein said radium bromatum has been diluted thirty times on a scale of 1:100; and
wherein said carbo animalis has been diluted thirty times on a scale of 1:100.

4. A method for relieving fatigue symptoms or cancer treatment related fatigue symptoms or radiation fatigue symptoms, in a subject in need thereof, which comprises administering to the subject and effective amount of homeopathic mixture, wherein the homeopathic mixture comprises (a) cadmium sulphuratum, (b) x-ray, (c) phosphoricum acidum, (d) radium bromatum, and (e) carbo animalis.

5. The method of claim 1,
wherein said cadmium sulphuratum has been diluted not less than twelve times and not more than two hundred times, on a scale of 1:100;
wherein said x-ray has been diluted not less than three times and not more than thirty times, on a scale of 1:100;
wherein said phosphoricum acidum has been diluted not less than twelve times and not more than two hundred times, on a scale of 1:100;
wherein said radium bromatum has been diluted not less than twelve times and not more than two hundred times, on a scale of 1:100; and
wherein said carbo animalis has been diluted not less than twelve times and not more than two hundred times, on a scale of 1:100.

6. The method of claim 1,
wherein said cadmium sulphuratum has been diluted thirty times on a scale of 1:100;
wherein said x-ray has been diluted six times on a scale of 1:100;
wherein said phosphoricum acidum has been diluted thirty times on a scale of 1:100;
wherein said radium bromatum has been diluted thirty times on a scale of 1:100; and
wherein said carbo animalis has been diluted thirty times on a scale of 1:100.
